(19) United States
(12) Patent Application Publication
(10) Pub. No.: US 2020/0061036 A1
(43) Pub. Date: Feb. 27, 2020

(54) ACECLIDINE ISOMERS AND SCALEMIC MIXTURES THEREOF FOR THE TREATMENT OF PRESBYOPIA

(71) Applicant: Presbyopia Therapies, LLC, Coronado, CA (US)

(72) Inventor: Gerald Horn, Deerfield, IL (US)

(21) Appl. No.: 16/543,835

(22) Filed: Aug. 19, 2019

Related U.S. Application Data
(60) Provisional application No. 62/720,421, filed on Aug. 21, 2018.

(51) Int. Cl. A61K 31/439; A61P 27/10

(52) U.S. Cl. CPC A61K 31/439; A61P 27/10

(57) ABSTRACT

The invention provides compositions containing aceclidine isomers and scalemic mixtures thereof for the treatment of presbyopia. The compositions optionally contain an alpha-adrenergic agonist, a cycloplegic agent, a cryoprotectant, a non-ionic surfactant and/or a viscosity enhancer.

### FIELD OF THE INVENTION

The invention is directed to compositions containing aceclidine isomers and scalemic mixtures thereof for the treatment of presbyopia. The compositions optionally contain an alpha-adrenergic agonist, a cycloplegic agent, a cryoprotectant, a non-ionic surfactant, and/or a viscosity enhancer.

### BACKGROUND OF THE INVENTION

As a person ages the minimum distance from the eye at which an object will come into focus, provided distance vision is corrected or is excellent unaided, increases. For example, a 10-year-old can focus on an object or a “focal point” only three inches (0.072 meters) from their eye while still retaining excellent distance vision; a 40-year-old at six inches (0.15 meters); and a 60-year-old at an inconvenient 39 inches (1.0 meter). This condition of increasing minimum focal length in individuals with excellent unaided distance vision is called presbyopia, loosely translated as “old-man eye”.

Excellent unaided distance vision is also known as emmetropia. The inability to focus on distant focal points is known as myopia and the inability to focus on near focal points is known as hyperopia. Specifically, “distance” vision is considered any focal point 1 meter or more from the eye and near vision is any focal point less than 1 meter from the eye. The minimum focal length at which an object will come into focus is known as the “near point”. The change in focus from distance to the near point and any focal point in between is called accommodation. Accommodation is often measured in diopters. Diopters are calculated by taking the reciprocal of the focal length (in meters). For example, the decrease in accommodation from a 10-year-old eye to a 60-year-old eye is about 13 diopters (1÷0.072 meters=13.89 diopters; 1÷1 meter=1 diopter).

The highest incidence of first complaint of presbyopia occurs in people ages 42-44. Presbyopia occurs because as a person ages the eye's accommodative ability which uses near reflex-pupil constriction, convergence of the eyes and particularly ciliary muscle contraction, decreases. This reduction in accommodation results in an inadequate change in the normal thickening and increased curvature of the anterior surface of the lens that is necessary for the shift in focus from distant objects to near objects. Important near focus tasks affected by presbyopia include viewing computer screens (21 inches) and reading print (16 inches).

Presbyopia is a normal and inevitable effect of ageing and is the first unmistakable sign for many in their forties that they are getting older. One study found that more than 1 billion people worldwide were presbyopic in 2005. This same study predicted that number to almost double by the year 2050. If everyone over the age of 45 is considered to be presbyopic, then an estimated 122 million people in the United States alone had presbyopia in 2010. As baby boomers reach the critical age, this number is only going to increase.

Presbyopia carries with it a stigma resulting from the limitation in ability to quickly function at many tasks requiring focusing at both distant and near points, which once occurred almost immediately. In the presbyopic patient, these tasks can be performed only by the use of eyeglasses, contact lenses or after undergoing invasive surgery. One such optical modification, the monovision procedure, can be executed with the use of glasses, contact lenses or even surgery. The monovision procedure corrects one eye for near focus and the other eye for distance focus. However, monovision correction is normally accompanied by loss of depth perception and distance vision particularly in dim light (e.g. night). Other surgical procedures that have been developed to relieve presbyopia include: (1) the implantation of intraocular lenses (INTRACOR®; registered trademark of Technolas Perfect Vision GMBH); (2) reshaping of the cornea (PresbyLASIK and conductive keratoplasty); (3) scleral band expansion; and (4) implantation of corneal inlays (Flexivue Microlens®; registered trademark of PresbiBio LLC, Kamra®; registered trademark of AcuFocus, Inc. and Vue+). Kamra® corneal inlays manufactured by AcuFocus work by inlaying a pinhole on the cornea to increase the depth of focus.

A similar effect can be achieved with general miotic agents, such as pilocarpine (a non-selective muscarinic acetylcholine receptor agonist), carbachol (a non-selective muscarinic acetylcholine receptor agonist), and phospholine iodide (an acetylcholinesterase inhibitor). These general miotics can induce a pinhole pupil at sufficient concentrations to achieve pupils below 2.0 mm and potentially extend depth of focus much like an inlay, but at concentrations sufficient to cause pinhole pupil diameters of 2.0 mm or less these agents trigger increased ciliary muscle contraction and induce accommodation of any remaining reserves, improving near vision at the expense of distance vision in individuals who still retain some accommodative function. The side effects of ciliary spasm induced migraine like brow pain and blurred distance vision from induced myopia beyond the ability of a pinhole pupil to correct then necessitate using weaker concentrations with much shorter acting and more marginal effect, such as found with pilocarpine. In such cases even slight hyperopia helps offset the induced myopia while even very small increments of myopia, which is very common, exacerbate it. In extreme cases, such ciliary muscle spasms may possibly be associated with anterior chamber shallowing and pull on the ora serrata of the retina, resulting in a retinal tear and or retinal detachment.

Miotic agents have been described in various patent and patent applications for the treatment of presbyopia. U.S. Pat. Nos. 6,291,466 and 6,410,544 describe the use of pilocarpine to regulate the contraction of ciliary muscles to restore the eye to its resting state and potentially restore its accommodative abilities.

Miotics historically used to treat glaucoma, other than pilocarpine, particularly aceclidine, are also associated with ciliary spasm, brow and/or headache, and myopic blur. U.S. Pat. No. 9,833,441 describes the use of racemic aceclidine in combination with a cycloplegic agent to treat presbyopia with reduced side effects. U.S. Pat. No. 9,314,427 describes the use of racemic aceclidine and a cycloplegic agent to treat presbyopia while improving myopic blur (i.e. distance vision). However, it is unclear whether the use of scalemic mixtures of or enantiomerically pure aceclidine could extend the duration of presbyopic correction of or would suffer from the same side effects as racemic aceclidine.

Thus, there is a need in the art for a treatment for presbyopia containing a scalemic mixture of aceclidine or enantiomerically pure aceclidine that results in extended duration of effect and reduced side effects from the use of racemic aceclidine including myopic blur.

### SUMMARY OF THE INVENTION

The present invention is directed to compositions for the treatment of presbyopia comprising aceclidine wherein the S-enantiomer of aceclidine is greater than 50% by weight of the aceclidine, preferably greater than 60%, 70%, 80% or 90% of the aceclidine or preferably 100% of the aceclidine.

The present invention is further directed to compositions for the treatment of presbyopia comprising aceclidine wherein the R-enantiomer of aceclidine is greater than 50% by weight of the aceclidine, preferably greater than 60%, 70%, 80% or 90% of the aceclidine or preferably 100% of the aceclidine.

The present invention is further directed to methods of treating presbyopia comprising administering compositions of the present invention to a subject in need thereof.

### DETAILED DESCRIPTION OF THE INVENTION

It is a discovery of the present invention that an optimized scalemic mixture of or enantiomerically pure aceclidine can achieve an optimal degree of myopia (e.g. 1.0 diopter or less) and optimal pupil miosis to treat presbyopia while optimizing distance vision.

A pinhole depth of focus/field mechanism of action for topical presbyopia miotics has long been associated with pupillary miosis. Pupil size is often associated with accommodation. In addition, accommodation further enhances near vision to the extent full accommodative amplitude remains greater than 1.0 diopter (“D”) as is estimated to be the case for most presbyopes under the age of 65, and likely up to age 75 or beyond. For the latter to have optimal accommodative value, binocular instillation is preferred allowing the highest physiologic accommodation leading to improved depth perception. However, the degree of accommodation, which induces myopia, must be pinhole correctable by the pupil for optimized distance vision.

Pilocarpine for example induces −3 D to −11 D accommodation at 2% concentration in a somewhat age dependent manner, with −5 D or more having been documented to occur in presbyopes. Whereas, aceclidine induces much less accommodation, which has been found to be as little as −0.37 D, though with a range of variability. For distance vision to remain sharply focused without loss of 1 or more lines of vision, Pilocarpine requires both dilution to reduce the degree of induced myopia, and benefits from subjects that are not myopic. Mildly hyperopic subjects for example at +0.50 D emmetrope will neutralize −1 D of induced myopia to a net −0.50 and remain within the typical exponential decay curve with induced myopia of less than 1-line loss. Whereas for the same degree of myopia, a −0.50 D emmetrope will have a net −1.50 D refraction after −1 D of induced myopia and experience 5 or more lines of distance vision loss, typically to about 20.125 Snellen acuity. By reducing pilocarpine concentration to a range from 0.1% to 1.0%, reduced levels of induced myopia result, but with corresponding reduction in the degree of miosis.

The present invention discovers that by adjusting the ratio of S- and R-enantiomers of aceclidine an optimized scalemic mixture may be achieved. This optimized scalemic mixture or an enantiomerically pure aceclidine allows for increased pupil contraction and achieves accommodation that is optimized to remain within the preferred pupil size to achieve optical distance correction. Specifically, the preferred pinhole pupil size is a range from 1.5 to 2.2 millimolar (“mm”) or even more preferably from 1.6 to 1.9 mm. This pinhole pupil size achieves both depth of focus/field via pinhole optics for a prolonged duration and binocularity including a binocular summation from about 16 to about 18 inches to infinity. This pupil size also achieves greater near vision for post instillation for potentially 1 or more hours to a closer near point, such as 14″ to infinity, and potentially as close as 10 inches to infinity.

In one embodiment, the present invention is directed to compositions for the treatment of presbyopia comprising aceclidine wherein the S-enantiomer of aceclidine is greater than 50% by weight of the aceclidine, preferably greater than 60%, 70%, 80% or 90% of the aceclidine or preferably 100% of the aceclidine.

In another embodiment, the S-enantiomer of aceclidine may be present in compositions of the present invention at concentrations from about 0.1% to about 5.0% w/v.

In another embodiment, the present invention is further directed to compositions for the treatment of presbyopia comprising aceclidine wherein the R-enantiomer of aceclidine is greater than 50% by weight of the aceclidine, preferably greater than 60%, 70%, 80% or 90% of the aceclidine or preferably 100% of the aceclidine.

In another embodiment, the R-enantiomer of aceclidine may be present in compositions of the present invention at concentrations from about 0.1% to about 5.0% w/v.

In another embodiment, aceclidine may be present in compositions of the present invention at a ratio from about 9:1 to 1.1:1 S-enantiomer to R-enantiomer or 1:1.1 to about 1:9 S-enantiomer to R-enantiomer.

In another embodiment, the compositions of the present invention further comprise one or more excipients selected from the group consisting of an alpha-adrenergic agonist, a cycloplegic agent, a cryoprotectant, a non-ionic surfactant and a viscosity agent.

Alpha-adrenergic agonists suitable for use in the present invention include, but are not limited to, oxymetazoline, brimonidine, fadolmidine, phenylephrine, guanfacine or a combination thereof.

Alpha-adrenergic agonists may be present in compositions of the present invention at concentrations from about 0.01% to about 2.0% w/v.

Cycloplegic agents suitable for use in the present invention include, but are not limited to, tropicamide, atropine, Cyclogyl® (cyclopentolate hydrochloride), hyoscine, pirenzepine, 4-diphenylacetoxy-N-methylpiperidine methobromide (4-DAMP), AF-DX 384, methoctramine, tripitramine, darifenacin, solifenacin (Vesicare), tolterodine, oxybutynin, ipratropium, oxitropium, tiotropium (Spriva), otenzepad (a.k.a. AF-DX 116 or 11-{[2-(diethylamino)methyl]-1-piperidinyl}acetyl]-5,11-dihydro-6H-pyrido[2,3b][1,4]benzodiazepine-6-one) or a combination thereof.

Cycloplegic agents may be present in compositions of the present invention at concentrations from about 0.004% to about 0.05% w/v. In a preferred embodiment the cycloplegic agent is tropicamide at a concentration from about 0.004% to about 0.025% w/v.

Cryoprotectants are compounds that either prevent freezing or prevent damage to compounds during freezing. As used herein, the term “cryoprotectant” or “cryoprotectants” include lyoprotectants. Cryoprotectants suitable for use in the subject invention include, but are not limited to, a polyol, a sugar, an alcohol, a lower alkanol, a lipophilic solvent, a hydrophilic solvent, a bulking agent, a solubilizer, a surfactant, an antioxidant, a cyclodextrin, a maltodextrin, colloidal silicon dioxide, polyvinyl alcohol, glycine, 2-methyl-2,4-pentanediol, cellobiose, gelatin, polyethylene glycol (PEG), dimethyl sulfoxide (DMSO), formamide, antifreeze protein 752 or a combination thereof.

In one embodiment, the present invention individually excludes each cryoprotectant from the definition of cryoprotectant.

As used herein the term “polyol” refers to compounds with multiple hydroxyl functional groups available for organic reactions such as monomeric polyols such as glycerin, pentaerythritol, ethylene glycol and sucrose. Further, polyols may refer to polymeric polyols including glycerin, pentaerythritol, ethylene glycol and sucrose reacted with propylene oxide or ethylene oxide. In a preferred embodiment, polyols are selected from the group consisting of mannitol, glycerol, erythritol, lactitol, xylitol, sorbitol, isosorbide, ethylene glycol, propylene glycol, maltitol, threitol, arabitol and ribitol. In a more preferred embodiment, the polyol is mannitol.

Sugars suitable for use in the present invention as cryoprotectants include, but are not limited to, glucose, sucrose, trehalose, lactose, maltose, fructose and dextran.

In another preferred embodiment, alcohols include, but are not limited to, methanol.

As used herein “lower alkanols” include C1-C6 alkanols. Lower alkanols, suitable for use in the present invention include, but are not limited to, amyl alcohol, butanol, sec-butanol, t-butyl alcohol, n-butyl alcohol, ethanol, isobutanol, methanol. isopropanol and propanol.

Bulking agents suitable for use in the present invention include, but are not limited to, saccharide, polyvinylpyrrolidone, cyclodextrin and trehalose.

Solubilizers suitable for use in the present invention include, but are not limited to, cyclic amide, gentisic acid and cyclodextrins.

Cryoprotectants may be at present in compositions of the present invention at a concentration from about 0.1% to about 99% w/v, preferably from about 0.5% to about 50% w/v, more preferably from about 0.5% to about 10% w/v. In a preferred embodiment, the cryoprotectant is a polyol at a concentration from about 0.5% to about 4.0% w/v, more preferably mannitol at a concentration from about 2.0% to about 4.0% w/v.

Non-ionic surfactants, suitable for use in the present invention include, but are not limited to, cyclodextrins, polyoxyl alkyls, polysorbates, poloxamers or a combination thereof including poloxamer 103, poloxamer 123, poloxamer 124, poloxamer 108, poloxamer 188, poloxamer 338, poloxamer 407, polysorbate 20, polysorbate 40, polysorbate 60, polysorbate 80, ionically charged (e.g. anionic) beta-cyclodextrins with or without a butyrated salt (Captisol®) 2-hydroxypropyl beta cyclodextrin (“HPβCD”), alpha cyclodextrins, gamma cyclodextrins, cyclodextrin, hydroxypropyl-β-cyclodextrin, hydroxypropyl-γ-cyclodextrin, randomly methylated β-cyclodextrin, β-cyclodextrin sulfobutyl ether, γ-cyclodextrin sulfobutyl ether or glucosyl-β-cyclodextrin, polyoxyethylene, polyoxypropylene glycol, polyoxyl 35 castor oil, polyoxyl 40 hydrogenated castor oil, polyoxyethylene hydrogenated castor oil 60, polyoxyethylene (200), polyoxypropylene glycol (70), polyoxyethylene hydrogenated castor oil, polyoxyethylene hydrogenated castor oil 60, polyoxyl, polyoxyl stearate, nonoxynol, octyphenol ethoxylates, nonyl phenol ethoxylates, capryols, lauroglycol, polyethylene glycol (“PEG”), Brij® 35, 78, 98, 700 (polyoxyethylene glycol alkyl ethers), glyceryl laurate, lauryl glucoside, decyl glucoside, or cetyl alcohol; or zwitterion surfactants such as palmitoyl carnitine, cocamide DEA, cocamide DEA derivatives cocamidopropyl betaine, or trimethyl glycine betaine, N-2(2-acetamido)-2-aminoethane sulfonic acid (ACES), N-2-acetamido iminodiacetic acid (ADA), N,N-bis(2-hydroxyethyl)-2-aminoethane sulfonic acid (BES), 2-[Bis-(2-hydroxyethyl)-amino]-2-hydroxymethyl-propane-1,3-diol (Bis-Tris), 3-cyclohexylamino-1-propane sulfonic acid (CAPS), 2-cyclohexylamino-1-ethane sulfonic acid (CHES), N,N-bis(2-hydroxyethyl)-3-amino-2-hydroxypropane sulfonic acid (DIPSO), 4-(2-hydroxyethyl)-1-piperazine propane sulfonic acid (EPPS), N-2-hydroxyethylpiperazine-N′-2-ethane sulfonic acid (HEPES), 2-(N-morpholino)-ethane sulfonic acid (MES), 4-(N-morpholino)-butane sulfonic acid (MOBS), 2-(N-morpholino)-propane sulfonic acid (MOPS), 3-morpholino-2-hydroxypropanesulfonic acid (MOPSO), 1,4-piperazine-bis-(ethane sulfonic acid) (PIPES), piperazine-N,N′-bis(2-hydroxypropane sulfonic acid) (POPSO), N-tris(hydroxymethyl)methyl-2-aminopropane sulfonic acid (TAPS), N-[tris(hydroxymethyl)methyl]-3-amino-2-hydroxypropane sulfonic acid (TAPSO), N-tris(hydroxymethyl)methyl-2-aminoethane sulfonic acid (TES), 2-Amino-2-hydroxymethyl-propane-1,3-diol (Tris), tyloxapol, SolulanTM C-24 (2-[[10,13-dimethyl-17-(6-methylheptan-2-yl)-2,3,4,7,8,9,11,12,14,15,16,17-dodecahydro-1H-cyclopenta[a]phenanthren-3-yl]oxy]ethanol) and Span® 20-80 (sorbitan monolaurate, sorbitan monopalmitate, sorbitan monostearate, and sorbitan monooleate).

Preferably, the non-ionic surfactants used in the present invention achieve submicron diameter micelles, more preferably less than 200 nanometers and more preferably less than 150 nanometers in diameter.

Non-ionic surfactants may be present in compositions of the present invention at concentrations from about 0.5% to about 10% w/v. In a preferred embodiment the non-ionic surfactant is polysorbate 80, preferably at a concentration from about 1% to about 6% w/v and more preferably from about 1% to about 5% w/v, yet more preferably from about 2.5% to about 4% w/v and most preferably at about 2.5% or 2.75% or 3% or 4% or 5% w/v.

Ophthalmological in situ gels which may be substituted for or added in addition to one or more non-ionic surfactants include but are not limited to gelatin, carbomers of various molecular weights including carbomer 934 P and 974 P, xanthan gums, alginic acid (alginate), guar gums, locust bean gum, chitosan, pectins and other gelling agents well known to experts in the art.

Viscosity agents, suitable for use in the present invention include, but are not limited to, gums such as guar gum, hydroxypropyl-guar (“hp-guar”), and xanthan gum, alginate, chitosan, gelrite, hyaluronic acid, dextran, Carbopol® (polyacrylic acid or carbomer) including Carbopol® 900 series including Carbopol® 940 (carbomer 940), Carbopol® 910 (carbomer 910) and Carbopol® 934 (carbomer 934), cellulose derivatives such as carboxymethyl cellulose (“CMC”), methylcellulose, methyl cellulose 4000, hydroxymethyl cellulose, hydroxypropyl cellulose, hydroxypropylmethyl cellulose, hydroxyl propyl methyl cellulose 2906, carboxypropylmethyl cellulose, hydroxypropylethyl cellulose, and hydroxyethyl cellulose, polyethylene glycol, polyvinyl alcohol, polyvinyl pyrrolidone, gellan, carrageenan, alignic acid, carboxyvinyl polymer or a combination thereof.

In a preferred embodiment the viscosity agent will have an equilibration viscosity less than 100 cps, preferably from about 15 to about 35 cps, and most preferably at about 30 cps.

Viscosity agents may be present in compositions of the present invention at a concentration from about 0.05% to about 5.0%. In another preferred embodiment, the viscosity enhancer is hydroxypropylmethyl cellulose at a concentration from about 0.5% to about 1.75%, and more preferably about 0.75% or 1.5% and still more preferably from about 1.0% to about 1.5%.

In another embodiment, the compositions of the present invention further comprise one or more preservatives.

Preservatives, suitable for use in the present invention include, but are not limited to, benzalkonium chloride (“BAK”), sorbic acid, oxychloro complex, citric acid, chlorobutanol, thimerosal, phenylmercuric acetate, disodium ethylenediaminetetraacetic acid, dicalcium diethylenetriamine pentaacetic acid (“Ca2DTPA”), phenylmercuric nitrate, perborate or benzyl alcohol.

Preservatives may be present in compositions of the present invention at concentrations from about 0.001% to about 1.0% w/v. In a preferred embodiment the preservative is a combination of BAK, sorbic acid and disodium ethylenediaminetetraacetic acid.

Various buffers and means for adjusting pH can be used to prepare ophthalmological compositions of the invention. Such buffers include, but are not limited to, acetate buffers, citrate buffers, phosphate buffers and borate buffers. It is understood that acids or bases can be used to adjust the pH of the composition as needed, preferably of 1 to 10 mM concentration, and more preferably about 3 mM or 5 mM. In a preferred embodiment the pH is from about 4.0 to about 8.0, in a more preferred embodiment the pH is from about 5.0 to about 7.0.

In another embodiment, the present invention does not comprise timolol.

The present invention is further directed to methods of treating presbyopia comprising administering compositions of the present invention to a subject in need thereof.

The present invention is further directed to a method of treating presbyopia comprising administering compositions of the present invention to a subject in need thereof, wherein distance vision of the subject is either unaffected or improved.

As used herein, the term “composition” is intended to encompass a product comprising the specified ingredients in the specified amounts, as well as any product which results, directly or indirectly, from a combination of the specified ingredients in the specified amounts.

As used herein, all numerical values relating to amounts, weights, and the like, that are defined as “about” each particular value is plus or minus 10%. For example, the phrase “about 5% w/v” is to be understood as “4.5% to 5.5% w/v.” Therefore, amounts within 10% of the claimed value are encompassed by the scope of the claims.

As used herein “% w/v” refers to the percent weight of the total volume of the composition.

As used herein the term “subject” refers but is not limited to a person or other animal.

The term “aceclidine” encompasses its salts, esters, analogues, prodrugs and derivatives including, highly M1 selective 1,2,5 thiadiazole substituted analogues like those disclosed in Ward. J. S. et al., 1,2,5-Thiadiazole analogues of aceclidine as potent ml muscarinic agonists, J Med Chem, 1998, Jan. 29, 41(3), 379-392 and aceclidine prodrugs including but not limited to carbamate esters.

The term “brimonidine” encompasses, without limitation, brimonidine salts and other derivatives, and specifically includes, but is not limited to, brimonidine tartrate, 5-bromo-6-(2-imidazolin-2-ylamino)quinoxaline D-tartrate, and Alphagan®.

The terms “treating” and “treatment” refer to reversing, alleviating, inhibiting, or slowing the progress of the disease, disorder, or condition to which such terms apply, or one or more symptoms of such disease, disorder, or condition.

The term “pharmaceutically acceptable” describes a material that is not biologically or otherwise undesirable (i.e. without causing an unacceptable level of undesirable biological effects or interacting in a deleterious manner).

As used herein, the term “pharmaceutically effective amount” refers to an amount sufficient to effect a desired biological effect, such as a beneficial result, including, without limitation, prevention, diminution, amelioration or elimination of signs or symptoms of a disease or disorder. Thus, the total amount of each active component of the pharmaceutical composition or method is sufficient to show a meaningful subject benefit. Thus, a “pharmaceutically effective amount” will depend upon the context in which it is being administered. A pharmaceutically effective amount may be administered in one or more prophylactic or therapeutic administrations.

The term “prodrugs” refers to compounds, including, but not limited to, monomers and dimers of the compounds of the invention, which have cleavable groups and become, under physiological conditions, compounds which are pharmaceutically active in vivo.

As used herein “salts” refers to those salts which retain the biological effectiveness and properties of the parent compounds and which are not biologically or otherwise harmful at the dosage administered. Salts of the compounds of the present inventions may be prepared from inorganic or organic acids or bases.

The compounds of the present invention can be used in the form of pharmaceutically acceptable salts derived from inorganic or organic acids or bases. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq.

The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, hyaluronic acid, malic acid, sulphuric acid and phosphoric acid and such organic acids as oxalic acid, malic acid, maleic acid, methanosulfonic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethyl ammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

The term “ester” as used herein is represented by the formula —OC(O)A1 or —C(O)OA1, where A1 can be alkyl, cycloalkyl, alkenyl, cycloalkenyl, alkynyl, cycloalkynyl, aryl, a heteroaryl group or another suitable substituent.

The following examples are provided solely for illustrative purposes and are not meant to limit the invention in any way.

### EXAMPLES

### Example 1—Effect of Aceclidine Enantiomer Ratios (Prophetic)

### Method

Solutions containing scalemic mixtures of or enantiomerically pure aceclidine in 1.50% w/v polysorbate 80, 1.2% w/v hydroxypropylmethyl cellulose, 0.1% w/v disodium ethylenediaminetetraacetic acid, 0.1% w/v sorbate in 0.9% w/v sodium chloride buffered saline at a pH of 6.5 were instilled, binocularly in a subject. The subject's visual acuity at 45 centimeters was then tested both monocularly and binocularly. Net gain in lines of visual acuity versus baseline monocularly/binocularly are shown in Table 1 below at 1, 2 and 5 hours post-instillation.

### Results

TABLE 1
Net gain in lines of visual acuity

| S | R | 1 hour | 2 hours | 5 hours |
|---|---|---|---|---|
| 25 | 75 | 1.50/1.50 | 0.75/1.25 | 0.50/1.00 |
| 50 | 50 | 2.00/3.00 | 1.50/2.50 | 1.00/1.50 |
| 62.5 | 22.5 | 1.40/3.20 | 1.70/2.70 | 1.20/1.60 |
| 75 | 25 | 1.50/2.50 | 1.20/2.80 | 0.70/2.00 |
| 87.5 | 12.5 | 1.75/3.25 | 1.40/2.90 | 0.90/2.25 |
| 100 | 0 | 2.00/3.50 | 1.75/3.00 | 1.50/2.50 |

As shown in Table 1, the ratio of S- to R-enantiomer of aceclidine positively correlated with increased binocular visual acuity at 5 hours post-instillation. This results demonstrates that compositions containing scalemic mixtures of aceclidine containing greater than 50% of the S-enantiomer or enantiomerically pure S-aceclidine have superior presbyopic correction to racemic aceclidine.

### Example 2. Maintaining Aceclidine Presbyopic Effect at Various S/R Enantiomer Ratios (Prophetic)

For any given clinically effective concentration of aceclidine there exist various other concentrations of aceclidine that will give similar clinical results if the ratio of the S to R enantiomer is varied.

A composition comprising 1.75% racemic aceclidine, 2.5% mannitol, 0.1.0% EDTA, NaCl 0.90%, polysorbate 4.0%, and sorbate 0.10% was instilled to the eye of a presbyopic subject and resulted in 3 lines of vision correction. The following table represents the approximate change in concentration for equivalence of effect, both miosis and depth of field (near vision), based on various S to R enantiomer ratios.

TABLE 2
Efficacy Equivalent Aceclidine Concentrations for Various S to R Ratios

| | Total | % | | Near Vision |
|---|---|---|---|---|
| S to R | Aceclidine | Aceclidine | Miosis | Lines of |
| Ratio | Concentration | in S form | (mm) | Improvement |
| 1:7 | 5.25% | 0.66% | 1.5 | 3 |
| 1:3 | 2.63% | 0.66% | 1.5 | 3 |
| 1:1.67 | 2.04% | 0.77% | 1.5 | 3 |
| 1:1 | 1.75% | 0.88% | 1.5 | 3 |
| 1.67:1 | 1.17% | 0.73% | 1.5 | 3 |
| 3:1 | 0.93% | 0.70% | 1.5 | 3 |
| 7:1 | 0.88% | 0.77% | 1.5 | 3 |
| 1:0 | 0.58% | 0.58% | 1.5 | 3 |

As shown in Table 2, a decrease in the ratio of S to R enantiomer of aceclidine requires an increase in the total concentration of aceclidine to achieve similar presbyopic correction. Whereas an increase in the ratio of the S to R enantiomer of aceclidine requires a decrease in the total concentration of aceclidine to achieve similar presbyopic correction. However, this change in total concentration does not exactly correlate with the total concentration of the S enantiomer in the composition. This lack of correlation is most apparent in the 1:0 ratio where only 0.58% of the S enantiomer of aceclidine is needed to reproduce the presbyopic effect of 0.88% of the S enantiomer in a 1:1 ratio.

What is claimed is:

1. A composition for the treatment of presbyopia comprising aceclidine wherein the S-enantiomer of aceclidine is greater than 50% by weight of the aceclidine.

2. The composition of claim 1, wherein the S-enantiomer is at least 60% by weight of the mixture.

3. The composition of claim 1, wherein the S-enantiomer is at least 70% by weight of the aceclidine.

4. The composition of claim 1, wherein the S-enantiomer is at least 80% by weight of the aceclidine.

5. The composition of claim 1, wherein the S-enantiomer is at least 90% by weight of the aceclidine.

6. The composition of claim 1, wherein the S-enantiomer is at least 99% by weight of the aceclidine.

7. The composition of claim 1, wherein the S-enantiomer is 100% by weight of the aceclidine.

8. The composition of claim 1, further comprising one or more agents selected from the group consisting of an alpha-adrenergic agonist, a cycloplegic agent, a cryoprotectant, a non-ionic surfactant and a viscosity agent.

9. The composition of claim 1, further comprising one or more preservatives.

10. A method of treating presbyopia comprising administering the composition of claim 1 to a subject in need thereof.

11. A composition for the treatment of presbyopia comprising aceclidine wherein the R-enantiomer of aceclidine is greater than 50% by weight of the aceclidine.

12. The composition of claim 10, wherein the R-enantiomer is at least 60% by weight of the mixture.

13. The composition of claim 1, wherein the R-enantiomer is at least 70% by weight of the aceclidine.

14. The composition of claim 10, wherein the R-enantiomer is at least 80% by weight of the aceclidine.

15. The composition of claim 10, wherein the R-enantiomer is at least 90% by weight of the aceclidine.

16. The composition of claim 10, wherein the R-enantiomer is at least 99% by weight of the aceclidine.

17. The composition of claim 10, wherein the R-enantiomer is 100% by weight of the aceclidine.

18. The composition of claim 10, further comprising one or more agents selected from the group consisting of an alpha-adrenergic agonist, a cycloplegic agent, a cryoprotectant, a non-ionic surfactant and a viscosity agent.

19. The composition of claim 10, further comprising one or more preservatives.

20. A method of treating presbyopia comprising administering the composition of claim 10 to a subject in need thereof.
